(19) United States
(12) Patent Application Publication
(10) Pub. No.: US 2025/0000413 A1
(43) Pub. Date: Jan. 2, 2025

(54) ASSISTANCE APPARATUS, ASSISTANCE METHOD, AND COMPUTER READABLE MEDIUM

(71) Applicant: Yokogawa Electric Corporation, Tokyo (JP)

(72) Inventors: Kiyomi MURAOKA, Tokyo (JP); Hirotaka YAMASHITA, Tokyo (JP); Takashi TSUBOTA, Tokyo (JP); Motohiko ISHIDA, Tokyo (JP)

(21) Appl. No.: 18/752,743

(22) Filed: Jun. 24, 2024

(30) Foreign Application Priority Data
Jun. 27, 2023 (JP) 2023-104703

(51) Int. Cl. A61B 5/16; A61B 5/374; A61B 5/384; G16H 40/67; G16H 50/20

(52) U.S. Cl. CPC A61B 5/165; A61B 5/374; A61B 5/384; G16H 40/67; G16H 50/20

(57) ABSTRACT

Provided is an assistance apparatus including an information acquisition unit which acquires brain wave information of a target person in a state in which dialog is difficult in a case where a living subject conducts an engagement for the target person, and a determination unit which determines a state of the target person based on the brain wave information. The information acquisition unit acquires biological information of the target person. The determination unit determines the state of the target person based on the brain wave information and the biological information. The information acquisition unit acquires the brain wave information of the target person before the engagement. The determination unit generates state information indicating the state of the target person based on a change of the brain wave information before and after the engagement and the biological information, and determines the state of the target person based on the state information.

### BACKGROUND

### 1. Technical Field

The present invention relates to an assistance apparatus, an assistance method, and a computer readable medium.

### 2. Related Art

Patent document 1 describes “a reinforcement target behavior is reinforced without causing a user to be aware” (Abstract of the Disclosure). Patent document 2 describes “three-dimensional movement control of an alter ego of an operator in a VR space is achieved” (Abstract of the Disclosure). Patent document 3 describes “a feedback loop is generated in which a state of a brain of an individual modulates a parameter of an augmented reality system” (Abstract of the Disclosure).

### LIST OF CITED REFERENCES

### Patent Documents

Patent document 1: International Publication No. 2019/082687
Patent document 2: Japanese Patent Application Publication No. 2022-020057
Patent document 3: Japanese translation publication of a PCT route patent application No. 2021-511612

### BRIEF DESCRIPTION OF THE DRAWINGS

FIG. 1A illustrates an example of a situation before a living subject 120 conducts an engagement for a target person 110.

FIG. 1B illustrates an example of a situation in which the living subject 120 is conducting the engagement for the target person 110.

FIG. 2 is a block diagram illustrating an example of an assistance apparatus 100 according to an embodiment of the present invention.

FIG. 3 illustrates an example of an information acquisition unit 10.

FIG. 4 illustrates an example of state information Is.

FIG. 5A illustrates an example of learning by a state learning unit 40.

FIG. 5B illustrates an example of an inference by an engagement inference model 42.

FIG. 6A illustrates an example of a situation in which the living subject 120 is conducting an engagement En′1 for the target person 110.

FIG. 6B illustrates an example of a situation in which the living subject 120 is conducting an engagement En′2 for the target person 110.

FIG. 7 is a flowchart illustrating an example of an assistance method according to an embodiment of the present invention.

FIG. 8 illustrates an example of a computer 2200 in which the assistance apparatus 100 according to an embodiment of the present invention may be embodied entirely or in part.

### DESCRIPTION OF EXEMPLARY EMBODIMENTS

Hereinafter, the present invention will be described through embodiments of the invention, but the following embodiments do not limit the invention according to claims. In addition, not all combinations of features described in the embodiment are essential to the solution of the invention.

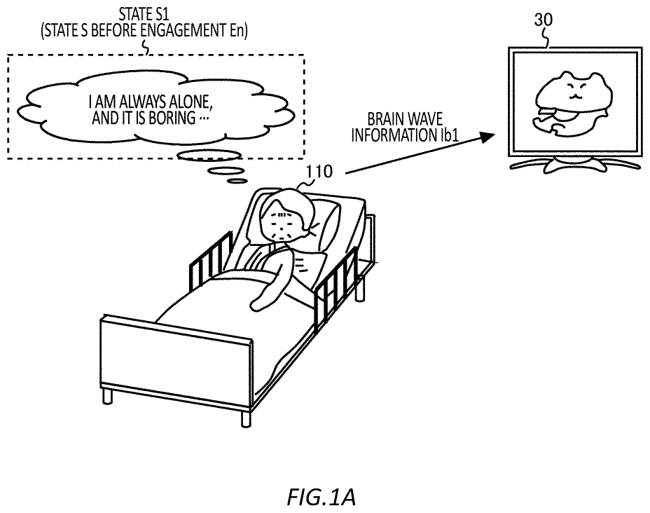

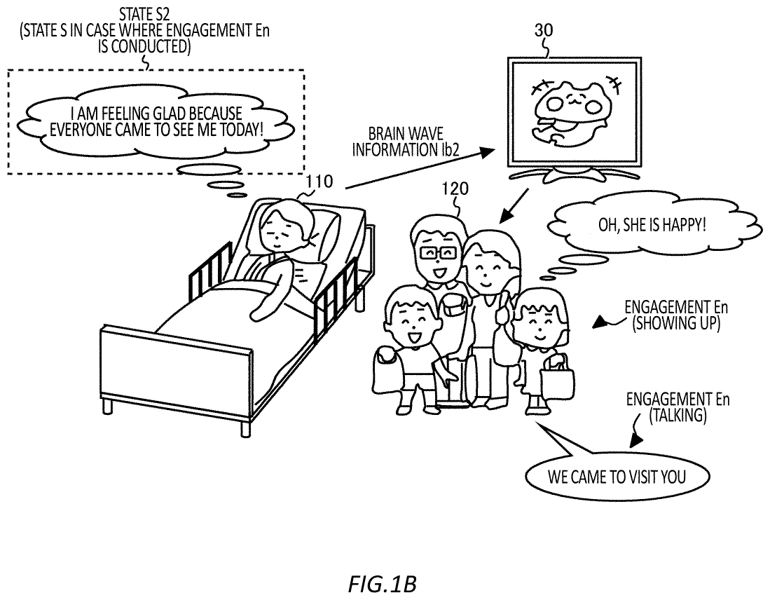

FIG. 1A illustrates an example of a situation before a living subject 120 conducts an engagement for a target person 110. FIG. 1B illustrates an example of a situation in which the living subject 120 is conducting the engagement for the target person 110. The target person 110 is in a state in which dialog is difficult due to a reason such being ill or having a disability, for example. The state in which the dialog is difficult may include a case where indication of an intention based on a body motion is difficult since the body motion is difficult. The living subject 120 is a living matter which may affect a potential state of the target person 110 by conducting an engagement for the target person 110. The living subject 120 may be a human being, or may be an animal such as a dog or a cat. In the present example, the living subject 120 is a human being.

An engagement conducted for the target person 110 is referred to as an engagement En. The engagement En may refer to a motion which affects the potential state of the target person 110 such as talking to the target person 110, showing up, or touching, or a motion for changing a surrounding environment of the target person 110 such as playing music or changing air conditioning. In the example of FIG. 1B, the living subject 120 that is a human being is talking to the target person 110 who is hospitalized, saying, “we came to visit you”.

A state of the target person 110 is set as an state S. The state S may be a potential state of the target person 110. The potential state of the target person 110 is their own psychological state that the target person 110 themselves is not aware of. A state presentation unit 30 presents the state S. The state presentation unit 30 may be a display, a monitor, or the like.

The state S of the target person 110 before the living subject 120 conducts the engagement En is set as a state S1. The state S of the target person 110 in a case where the living subject 120 conducts the engagement En is set as a state S2. The state S2 is the state S of the target person 110 after the living subject 120 has conducted the engagement En. The state S1 and the state S2 of the target person 110 are illustrated in broken line parts in FIG. 1A and FIG. 1B. In the example of FIG. 1A, before the living subject 120 conducts the engagement En, the target person 110 is feeling “I am always alone, and it is boring”. In the example of FIG. 1B, after the living subject 120 has conducted the engagement En, the target person 110 is feeling “I am glad because everyone came to see me today”. In the example of FIG. 1B, the engagement En is the living subject 120 that is a human being showing up themselves and talking to the target person 110, saying, “we came to visit you”.

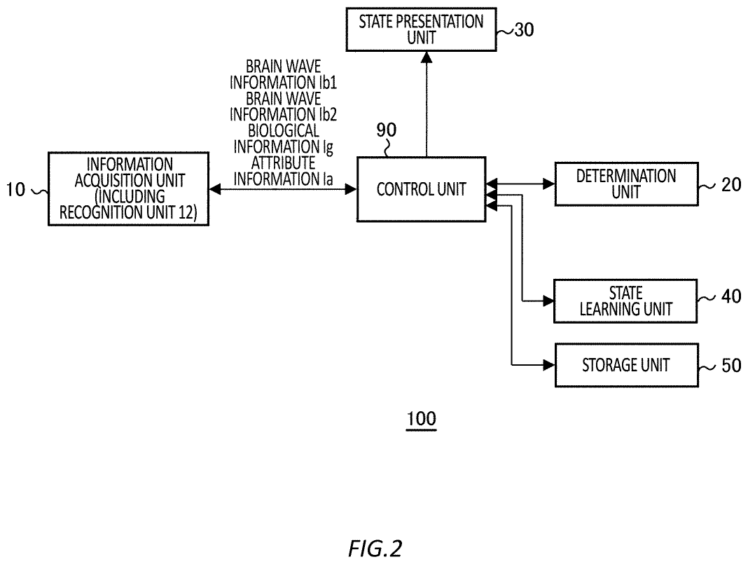

FIG. 2 is a block diagram illustrating an example of an assistance apparatus 100 according to an embodiment of the present invention. The assistance apparatus 100 includes an information acquisition unit 10 and a determination unit 20. The assistance apparatus 100 may include the state presentation unit 30, a state learning unit 40, a storage unit 50, and a control unit 90. The information acquisition unit 10 may include a recognition unit 12.

Part or whole of the assistance apparatus 100 may be achieved by a computer. The control unit 90 may be a central processing unit (CPU) of the computer. When the assistance apparatus 100 is achieved by a computer, a program for causing the computer to function as the assistance apparatus 100 may be installed in the computer, and a program for executing an assistance method which will be described below may be installed in the computer.

Brain wave information of the target person 110 is set as brain wave information Ib. The information acquisition unit 10 acquires the brain wave information Ib of the target person 110. The brain wave information Ib may be information for reproducing at least part of a temporal waveform of a brain wave of the target person 110. The brain wave information Ib may include data obtained by sampling the temporal waveform of the brain wave, may include data indicating a magnitude of a frequency component of a brain wave at one or more frequencies, and may include other data. For example, the brain wave information Ib includes data indicating a magnitude of a component of at least one of an alpha wave, a beta wave, a theta wave, a delta wave, or a gamma wave.

The alpha wave may be further classified into a high alpha wave, a medium alpha wave, and a low alpha wave depending on a frequency band. The beta wave may be classified into a high beta wave and a low beta wave. The brain wave information Ib may include data indicating a magnitude of at least one of the high alpha wave, the medium alpha wave, or the low alpha wave. The brain wave information Ib may include data indicating a magnitude of at least either the high beta wave or the low beta wave.

The brain wave information Ib may include information of a temporal waveform of one or more brain waves measured at one or more locations in a head part including the head and the face of the target person 110. For example, the brain wave information Ib may be acquired by measuring temporal waveforms of potentials of electrodes arranged at equal intervals in a vicinity of a scalp of the target person 110 as in an international 10-20 system, or may be acquired by another method. A configuration may be adopted where a plurality of electrodes arranged on the scalp are not equally spaced. The electrodes may be provided to wearable appliance to be worn on the head part of the target person 110 such as a headgear, headphones, earphones, or glasses. The brain wave information Ib may be information obtained by acquiring an electric signal at an electrode embedded in a body of the target person 110 through wireless communication. In the examples of FIG. 1A and FIG. 1B, the brain wave information Ib is wirelessly transmitted to the control unit 90.

The brain wave information Ib of the target person 110 when the target person 110 is in the state S1 is set as brain wave information Ib1 (see FIG. 1A). The brain wave information Ib of the target person 110 when the target person 110 is in the state S2 is set as brain wave information Ib2 (see FIG. 1B). The information acquisition unit 10 acquires the brain wave information Ib2. The information acquisition unit 10 may acquire the brain wave information Ib1. The determination unit 20 determines the state S2 of the target person 110 based on the brain wave information Ib2. The determination unit 20 may determine the state S1 of the target person 110 based on the brain wave information Ib1.

The state presentation unit 30 may present the state S determined by the determination unit 20. The state presentation unit 30 presents an avatar of the target person 110, for example, and presents the state S2 by way of a facial expression, a motion, or the like of the avatar. In the example of FIG. 1B, the state presentation unit 30 presents the state S2 in which the target person 110 is feeling “I am glad because everyone came to see me today”. As a result, the living subject 120 can recognize the state S2 of the target person 110 who has a difficulty in dialog. In the example of FIG. 1B, the living subject 120 is feeling “oh, she is happy” by recognizing the state S2 of the target person 110. In the example of FIG. 1B, the state S2 is presented by way of a facial expression of a virtual animal displayed on the state presentation unit 30.

The determination unit 20 may determine the state S1 or the state S2 based on a magnitude of a particular frequency component of the brain wave of the target person 110. The determination unit 20 may determine the state S1 or the state S2 based on a magnitude of one or more components among the alpha wave, the beta wave, the theta wave, the delta wave, and the gamma wave.

A sum of amplitudes of the alpha wave (8 Hz or more and less than 14 Hz), the beta wave (14 Hz or more and less than 26 Hz), the theta wave (4 Hz or more and less than 8 Hz), the gamma wave (26 Hz or more and less than 40 Hz), and the delta wave (less than 4 Hz) at certain timing is set as a total amplitude As. As an example, when a proportion of the amplitude of the delta wave of the target person 110 to the total amplitude As is greater than any of a proportion of the amplitude of the alpha wave to the total amplitude As, a proportion of the amplitude of the beta wave to the total amplitude As, a proportion of the amplitude of the theta wave to the total amplitude As, and a proportion of the amplitude of the gamma wave to the total amplitude As, it may be inferred that the target person 110 is in a sleeping state. As an example, when the proportion of the amplitude of the theta wave to the total amplitude As during a meeting of the target person 110 is greater than the proportion of the amplitude of the theta wave to the total amplitude As before the meeting of the target person 110, it may be inferred that fatigue or sleepiness of the target person 110 increases.

As an example, when a proportion of a sum of the amplitude of the low alpha wave (8 Hz or more and less than 10 Hz) and the amplitude of the medium alpha wave (10 Hz or more and less than 12 Hz) of the target person 110 to the total amplitude As increases over a lapse of time, it may be inferred that a degree of relaxation of the target person 110 increases.

As an example, when a proportion of a sum of the amplitude of the high alpha wave (12 Hz or more or less than 14 Hz) and the amplitude of the low beta wave (14 Hz or more and less than 18 Hz) of the target person 110 to the total amplitude As increases over a lapse of time, it may be inferred that a well balanced state between relaxation and concentration of the target person 110 increases. The well balanced state between relaxation and concentration is so called a preoccupied state.

It is a high probability that the degree of relaxation of the target person 110 is higher as the proportion of the sum of the amplitude of the low alpha wave and the amplitude of the medium alpha wave of the target person 110 to the total amplitude As is greater. Thus, the determination unit 20 may determine that the degree of relaxation of the target person 110 is higher as the proportion of the sum of the amplitude of the low alpha wave and the amplitude of the medium alpha wave to the total amplitude As in the brain wave information Ib2 is greater. It is a high probability that a degree of preoccupation of the target person 110 is higher as the proportion of the sum of the amplitude of the high alpha wave and the amplitude of the low beta wave of the target person 110 to the total amplitude As is greater. Thus, the determination unit 20 may determine that the degree of preoccupation of the target person 110 is higher as the proportion of the sum of the amplitude of the high alpha wave and the amplitude of the low beta wave to the total amplitude As in the brain wave information Ib2 is greater.

The determination unit 20 may determine that a sense of security of the target person 110 increases when the proportion of the sum of the amplitude of the low alpha wave and the amplitude of the medium alpha wave of the target person 110 to the total amplitude As after the engagement En is greater than the proportion of the sum of the amplitude of the low alpha wave and the amplitude of the medium alpha wave of the target person 110 to the total amplitude As before the engagement En. The determination unit 20 may evaluate that the degree of preoccupation of the target person 110 increases when the proportion of the sum of the amplitude of the high alpha wave and the amplitude of the low beta wave of the target person 110 to the total amplitude As after the engagement En is greater than the proportion of the sum of the amplitude of the high alpha wave and the amplitude of the low beta wave of the target person 110 to the total amplitude As before the engagement En.

The determination unit 20 may determine at least either the state S1 or the state S2 by combining a plurality of components among the alpha wave, the beta wave, the theta wave, the delta wave, and the gamma wave of the target person 110. For example, it is a high probability that the degree of relaxation of the target person 110 is higher as a value obtained by dividing a magnitude of the alpha wave of the target person 110 by a magnitude of the beta wave is greater. Thus, the determination unit 20 may determine that the degree of relaxation of the target person 110 is higher as the value obtained by dividing the magnitude of the alpha wave of the target person 110 by the magnitude of the beta wave is greater.

The potential state S1 of the target person 110 may be reflected on the brain wave information Ib1. The potential state S2 of the target person 110 may be reflected on the brain wave information Ib2. The determination unit 20 determines the state S2 of the target person 110 based on the brain wave information Ib2. The assistance apparatus 100 can determine the potential state S2 of the target person 110. When the state presentation unit 30 presents the state S2, the living subject 120 can recognize the state S2 of the target person 110.

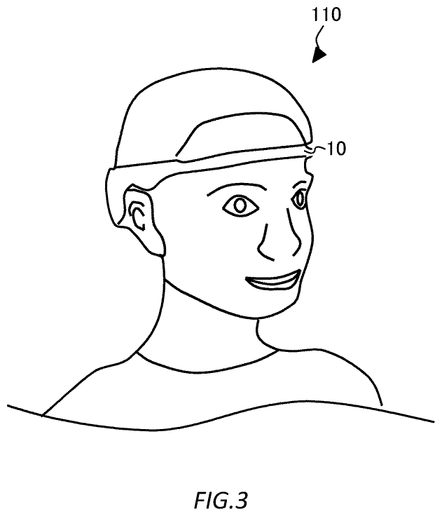

FIG. 3 illustrates an example of the information acquisition unit 10. The information acquisition unit 10 may have an electroencephalography which can measure the brain wave information Ib, or may have a communication device which acquires the brain wave information Ib measured by an external electroencephalography. The information acquisition unit 10 of the present example is an electroencephalography of a headgear type. The information acquisition unit 10 may be an electroencephalography of an earphone type. In the present example, the living subject 120 conducts the engagement En for the target person 110 in a state in which an electroencephalography of the headgear type or the earphone type is worn. As a result, the information acquisition unit 10 acquires the brain wave information Ib2 in a case where the engagement En is conducted for the target person 110.

The determination unit 20 and the control unit 90 (see FIG. 2) may be or may not be accommodated in a housing of the electroencephalography of the headgear type. When the determination unit 20 and the control unit 90 are not accommodated in the housing, the brain wave information Ib2 acquired by the information acquisition unit 10 may be wirelessly transmitted to the control unit 90.

The state presentation unit 30 may be or may not be accommodated in a housing of a headgear illustrated in FIG. 3. When the state presentation unit 30 is not accommodated in the housing of the headgear, the state presentation unit 30 may be a display, a monitor, or the like installed separately from the housing of the headgear. Information according to the state S1 or the state S2 may be wirelessly transmitted to the state presentation unit 30.

Biological information of the target person 110 is set as biological information Ig. The biological information Ig may include at least one of heartbeat information, perspiration amount information, or body temperature information of the target person 110. The biological information Ig of the target person 110 may be acquired by a sensor provided in wearable appliance (electroencephalography of the headgear type illustrated in FIG. 3, for example) which is worn by the target person 110.

The information acquisition unit 10 (see FIG. 2) may further acquire the biological information Ig of the target person 110. The information acquisition unit 10 may acquire the biological information Ig in a case where the living subject 120 conducts the engagement En for the target person 110. The determination unit 20 (see FIG. 2) may determine the state S2 of the target person 110 based on the brain wave information Ib2 and the biological information Ig. The potential state S of the target person 110 is likely to be reflected on the biological information Ig. For example, when the target person 110 is feeling stressed, the target person 110 is likely to be put into a state in which a sympathetic nerve is dominant over a parasympathetic nerve. When the sympathetic nerve is dominant over the parasympathetic nerve, a heartbeat fluctuation of the target person 110 is likely to decrease, and the perspiration amount is likely to increase. Thus, the determination unit 20 can appropriately determine the state S2 of the target person 110 based on the brain wave information Ib and the biological information Ig.

The determination unit 20 (see FIG. 2) may generate state information Is based on the brain wave information Ib and the biological information Ig. The state information Is refers to information based on the potential state S of the target person 110. The determination unit 20 may generate the state information Is based on a change from the brain wave information Ib1 to the brain wave information Ib2 and the biological information Ig. The determination unit 20 may determine the state S of the target person 110 based on the state information Is.

A magnitude of a first power spectrum in the heartbeat of the target person 110 is set as LF, and a magnitude of a second power spectrum is set as HF. A frequency band of the second power spectrum is a higher frequency band than a frequency band of the first power spectrum. A configuration may be adopted where the frequency band of the first power spectrum is not overlapped with the frequency band of the second power spectrum. The frequency band of the first power spectrum is, for example, 0.04 to 0.15 Hz. The frequency band of the second power spectrum is, for example, 0.15 to 0.4 Hz.

A change from a proportion of amplitudes of the high beta wave (18 or more and less than 26 Hz) and the gamma wave in the brain wave information Ib1 to the total amplitude As to a proportion of amplitudes of the high beta wave and the gamma wave in the brain wave information Ib2 to the total amplitude As is set as a change C1. The determination unit 20 may generate the state information Is based on the change C1 and a ratio (LF/HF) of LF to HF.

As an example, when a proportion of a sum of the amplitude of the high beta wave and the amplitude of the gamma wave of the target person 110 to the total amplitude As after the engagement En is greater than the proportion of the sum of the amplitude of the high beta wave and the amplitude of the gamma wave of the target person 110 to the total amplitude As before the engagement En and also the ratio (LF/HF) of LF to HF after the engagement En is equal to or greater than a threshold, it may be inferred that an irritated state, a nervous state, or a stressed state of the target person 110 increases. In this case, the determination unit 20 (see FIG. 2) may determine that the irritated state, the nervous state, or the stressed state of the target person 110 increases.

When the ratio (LF/HF) of LF to HF is equal to or greater than the threshold, it may be determined that the target person 110 is in a state in which the sympathetic nerve is dominant over the parasympathetic nerve. When the ratio (LF/HF) of LF to HF is less than the threshold, it may be determined that the target person 110 is in a state in which the parasympathetic nerve is dominant over the sympathetic nerve. The threshold may be 2, may be 3, may be 4, or may be 5.

As an example, when the proportion of the sum of the amplitude of the high beta wave and the amplitude of the gamma wave of the target person 110 to the total amplitude As after the engagement En is greater than the proportion of the sum of the amplitude of the high beta wave and the amplitude of the gamma wave of the target person 110 to the total amplitude As before the engagement En and also the ratio (LF/HF) of LF to HF after the engagement En is less than the threshold, it may be inferred that an excited state of the target person 110 increases. In this case, the determination unit 20 (see FIG. 2) may determine that the excited state of the target person 110 increases.

The determination unit 20 may generate the state information Is based on a magnitude relationship between the ratio (LF/HF) of LF to HF after the engagement En and the threshold of the proportion of LF to HF and the change C1. The threshold may be previously set. When the proportion of the sum of the amplitude of the high beta wave and the amplitude of the gamma wave of the target person 110 to the total amplitude As after the engagement En is greater than the proportion of the sum of the amplitude of the high beta wave and the amplitude of the gamma wave of the target person 110 to the total amplitude As before the engagement En and also the ratio (LF/HF) of LF to HF after the engagement En is equal to or greater than the threshold, the determination unit 20 (see FIG. 2) may generate the state information Is indicating that a sense of alertness of the target person 110 increases. When the proportion of the sum of the amplitude of the high beta wave and the amplitude of the gamma wave of the target person 110 to the total amplitude As after the engagement En is greater than the proportion of the sum of the amplitude of the high beta wave and the amplitude of the gamma wave of the target person 110 to the total amplitude As before the engagement En and also the ratio (LF/HF) of LF to HF after the engagement En is less than the threshold, the determination unit 20 may generate the state information Is indicating that a degree of excitement of the target person 110 increases.

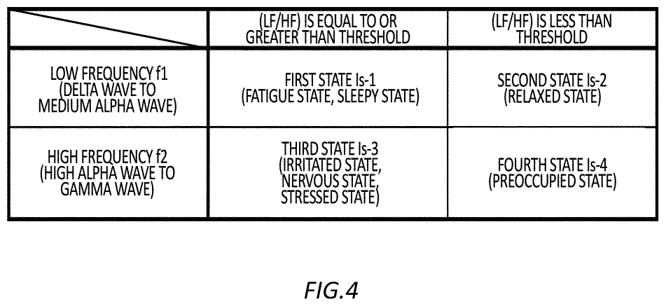

FIG. 4 illustrates an example of the state information Is. The state information Is may include information according to a plurality of states (a first state Is-1 to an n-th state Is-n) of the target person 110. In the present example, the state information Is includes information according to four states (the first state Is-1 to the fourth state Is-4) of the target person 110. In FIG. 4, a brain wave at a low frequency f1 refers to at least one of the delta wave, the theta wave, the low alpha wave, or the medium alpha wave, and a brain wave at a high frequency f2 refers to at least one of the high alpha wave, the low beta wave, the high beta wave, or the gamma wave.

An amplitude of the brain wave of the target person 110 that is an amplitude of the brain wave in a predetermined frequency band is set as an amplitude Af. The amplitude Af of the brain wave of the target person 110 before the engagement En is set as an amplitude Af1. The amplitude Af of the brain wave of the target person 110 after the engagement En is set as an amplitude Af2. The brain wave in the predetermined frequency band may be at least one of the low alpha wave, the medium alpha wave, the high alpha wave, the low beta wave, the high beta wave, the gamma wave, or the theta wave.

The determination unit 20 (see FIG. 2) may generate the state information Is based on a change from a proportion of the amplitude Af1 to the total amplitude As to a proportion of the amplitude Af2 to the total amplitude As and the ratio (LF/HF) of LF to HF. The state information Is may be the state information Is (any of the first state Is-1 to the n-th state Is-n) according to one state among the plurality of states of the target person 110.

In the present example, the first state Is-1 is a state of the target person 110 in a case where the proportion of the amplitude Af2 to the total amplitude As is greater than the proportion of the amplitude Af1 to the total amplitude As in the brain wave at the low frequency f1, and also the ratio (LF/HF) of LF to HF after the engagement En is equal to or greater than a threshold. When the target person 110 is in the first state Is-1, it may be inferred that the fatigue state or the sleepy state of the target person 110 increases. When the target person 110 is in the first state Is-1, the determination unit 20 (see FIG. 2) may generate the state information Is indicating that the fatigue state or the sleepy state of the target person 110 increases.

In the present example, the second state Is-2 is a state of the target person 110 in a case where the proportion of the amplitude Af2 to the total amplitude As is greater than the proportion of the amplitude Af1 to the total amplitude As in the brain wave at the low frequency f1, and also the ratio (LF/HF) of LF to HF after the engagement En is less than the threshold. When the target person 110 is in the second state Is-2, it may be inferred that a relaxed state of the target person 110 increases. When the target person 110 is in the second state Is-2, the determination unit 20 (see FIG. 2) may generate the state information Is indicating that a degree of relief of the target person 110 increases.

In the present example, the third state Is-3 is a state of the target person 110 in a case where the proportion of the amplitude Af2 to the total amplitude As is greater than the proportion of the amplitude Af1 to the total amplitude As in the brain wave at the high frequency f2, and also the ratio (LF/HF) of LF to HF after the engagement En is equal to or greater than the threshold. When the target person 110 is in the third state Is-3, it may be inferred that the irritated state, the nervous state, or the stressed state of the target person 110 increases. When the target person 110 is in the third state Is-3, the determination unit 20 (see FIG. 2) may generate the state information Is indicating that the irritated state, the nervous state, or the stressed state of the target person 110 increases.

In the present example, the fourth state Is-4 is a state of the target person 110 in a case where the proportion of the amplitude Af2 to the total amplitude As is greater than the proportion of the amplitude Af1 to the total amplitude As in the brain wave at the high frequency f2, and also the ratio (LF/HF) of LF to HF after the engagement En is less than the threshold. When the target person 110 is in the fourth state Is-4, it may be inferred that the preoccupied state of the target person 110 increases. When the target person 110 is in the fourth state Is-4, the determination unit 20 (see FIG. 2) may generate state information indicating that the preoccupied state of the target person 110 increases.

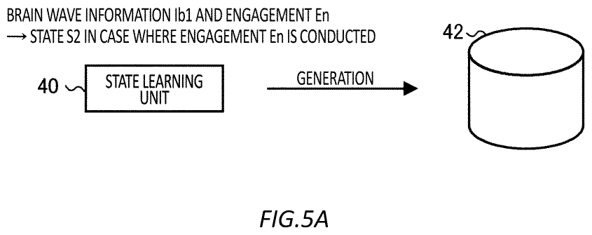

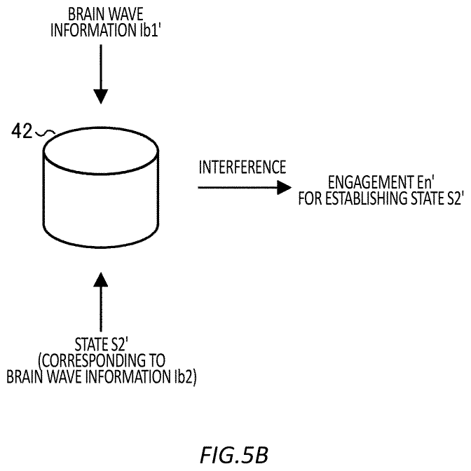

FIG. 5A illustrates an example of learning by the state learning unit 40. In a case where the target person 110 is put into the state S2 when the engagement En is conducted for the target person 110 with the brain wave information Ib1, the state learning unit 40 performs machine learning on a relationship between the brain wave information Ib1 and the engagement En and the state S2. The state learning unit 40 generates an engagement inference model 42 by performing the machine learning on the relationship between the brain wave information Ib1 and the engagement En and the state S2.

FIG. 5B illustrates an example of an inference by the engagement inference model 42. The engagement inference model 42 in FIG. 5B has already performed the machine learning on the relationship between the brain wave information Ib1 and the engagement En and the state S2. The brain wave information Ib1 of the target person 110 in an inference step illustrated in FIG. 5B is set as brain wave information Ib1′, and the predetermined state S2 of the target person 110 is set as a state S2′. The state S2′ may be a desired state of the target person 110 for the living subject 120. The desired state is, for example, the second state Is-2. The state S2′ may be a desired state for the target person 110.

The engagement inference model 42 makes an inference of an engagement En′ for putting the target person 110 into the state S2′ based on the brain wave information Ib1′ and the state S2′. Since the engagement inference model 42 has performed the machine learning on the relationship between the brain wave information Ib1 and the engagement En and the state S2, the engagement En for putting the target person 110 into the state S2′ may be inferred based on the brain wave information Ib1′ and the state S2′. As a result, a user of the assistance apparatus 100 can make an inference of the engagement En′ with a high probability with which the target person 110 can be put into the state S2′. The engagement inference model 42 may be stored in the storage unit 50 (see FIG. 2).

The state learning unit 40 (see FIG. 2) may perform machine learning on a relationship between the brain wave information Ib1 and the engagement En and the state S2 with regard to a plurality of living subjects 120. The state learning unit 40 may generate the engagement inference model 42 by performing the machine learning on the relationship between the brain wave information Ib1 and the engagement En and the state S2 with regard to the plurality of living subjects 120.

The assistance apparatus 100 may include the recognition unit 12 (see FIG. 2) which recognizes the living subject 120. The recognition unit 12 may be included in the information acquisition unit 10. The recognition unit 12 is, for example, an image capturing device, a microphone, or the like. When the recognition unit 12 is an image capturing device, the recognition unit 12 identifies one living subject 120 and another living subject 120 by captured images. When the recognition unit 12 is a microphone, the recognition unit 12 identifies one living subject 120 and another living subject 120 by a frequency of voice of the one living subject 120 and a frequency of voice of the another living subject 120.

The state learning unit 40 may perform the machine learning on the relationship between the brain wave information Ib1 and the engagement En and the state S2 with regard to the living subject 120 recognized by the recognition unit 12. The state learning unit 40 may generate the engagement inference model 42 for each of the living subjects 120.

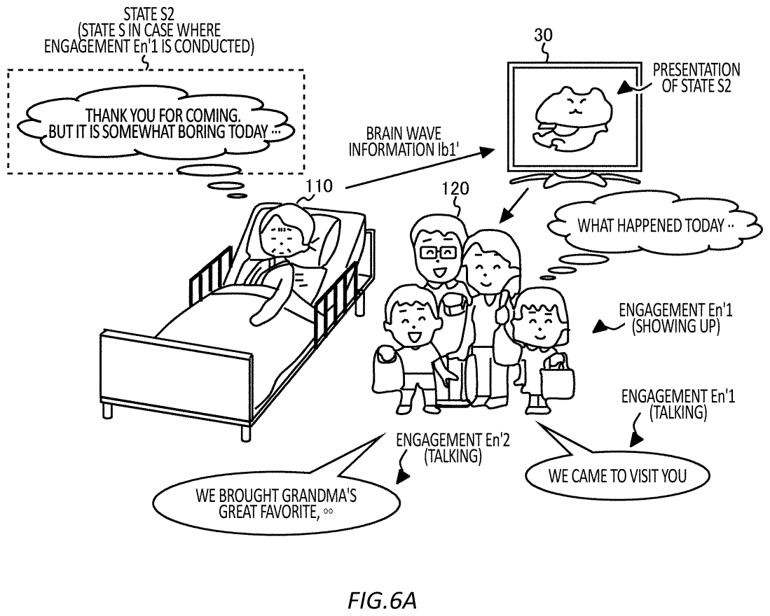

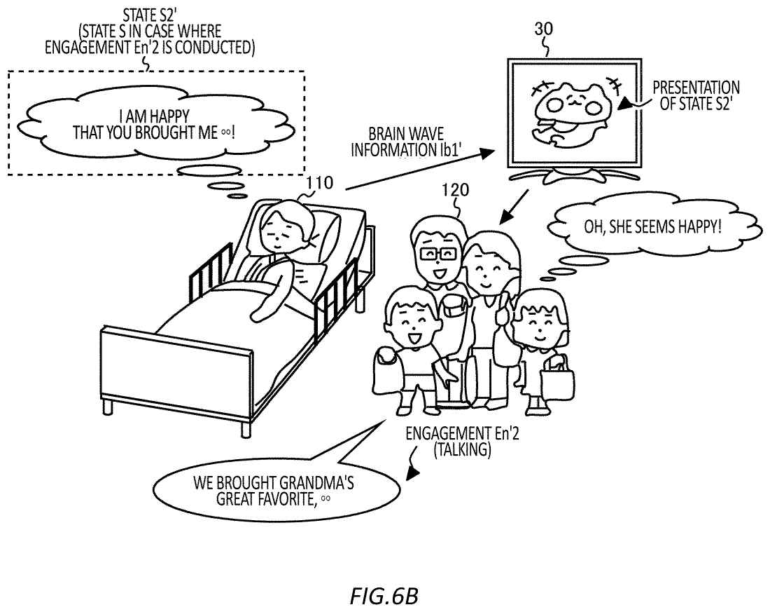

FIG. 6A illustrates an example of a situation in which the living subject 120 is conducting an engagement En′1 for the target person 110. FIG. 6B illustrates an example of a situation in which the living subject 120 is conducting an engagement En′2 for the target person 110. In the present example, the state learning unit 40 (see FIG. 2) performs the machine learning on the relationship between the brain wave information Ib1 and the engagement En and the state S2. In the present example, the engagement En′1 is the living subject 120 that is a human being showing up themselves and also talking to the target person 110, saying, “we came to visit you”. In the present example, the target person 110 to which the engagement En′1 is conducted is feeling “thank you for coming. But it is somewhat boring today . . . ”. The determination unit 20 determines this state S2 of the target person 110. In the present example, the state presentation unit 30 presents the state S2 by way of a facial expression of the virtual animal.

The state presentation unit 30 may present the engagement En′2 which may put the target person 110 into the state S2′. As a result, the living subject 120 can recognize the engagement En′ for putting the target person 110 into the predetermined state S2′. In the present example, the engagement En′2 is gifting a souvenir. In the present example, while gifting the souvenir, the living subject 120 is talking to the target person 110, saying, “we brought grandma's great favorite, ◯◯”. The state learning unit 40 (see FIG. 2) performs the machine learning on the relationship between the brain wave information Ib1 and the engagement En and the state S2. Thus, it is a high probability that the living subject 120 can put the target person 110 into the state S2′ by the engagement En′.

The information acquisition unit 10 (see FIG. 2) may acquire the brain wave information Ib2 after the engagement En′ has been conducted by the living subject 120. The determination unit 20 (see FIG. 2) may determine the state S2′ of the target person 110 based on the brain wave information Ib2. The state presentation unit 30 may present the state S2′ of the target person 110 determined by the determination unit 20 (see FIG. 2). In the present example, the state presentation unit 30 presents the state S2′ in which the target person 110 is feeling “I am happy that you brought me ◯◯”. As a result, the living subject 120 can recognize the state S2′ of the target person 110. In the present example, the living subject 120 is feeling “oh, she seems happy” by recognizing the state S2′ of the target person 110. In the present example, the state S2′ is presented by way of the facial expression of the virtual animal displayed on the state presentation unit 30. The living subject 120 can check whether the engagement En′ presented by the state presentation unit 30 is appropriate by recognizing the state S2′ of the target person 110.

The information acquisition unit 10 (see FIG. 2) may acquire attribute information indicating an attribute of the living subject 120. The attribute information is set as attribute information Ia. When the living subject 120 is a human being, the attribute information Ia may include information related to at least one of an age, a gender, an occupation, or a preference of the human being. The attribute information Ia may include information related to whether the living subject 120 is a family member of the target person 110. When the living subject 120 is a family member of the target person 110, the attribute information Ia may include information related to parents or the like of the target person 110 and the living subject 120.

The state learning unit 40 (see FIG. 2) may perform machine learning on a relationship among the attribute information Ia of the living subject 120, the brain wave information Ib1 and the engagement En by the living subject 120, and the state S2. The engagement inference model 42 makes an inference of the engagement En′ for putting the target person 110 into the state S2′ based on the brain wave information Ib1′, the state S2′, and the attribute information Ia. When the brain wave information Ib1′ of the target person 110 is one of the brain wave information Ib1, the engagement En′ for putting the target person 110 into the state S2′ may be different depending on the attribute of the living subject 120. Since the engagement inference model 42 has performed the machine learning on the relationship among the attribute information Ia, the brain wave information Ib1 and the engagement En, and the state S2, the engagement En for putting the target person 110 into the state S2′ may be inferred based on the brain wave information Ib1′, the state S2′, and the attribute information Ia.

The state presentation unit 30 may present the engagement En′ inferred by the engagement inference model 42 for each of attributes of the living subjects 120. For example, the state presentation unit 30 presents mutually different engagements En′ when the living subject 120 is a nurse and when the living subject 120 is a visitor of a friend of the target person 110 or the like. As a result, the living subject 120 can determine an optimal engagement En′ according to their own attribute.

When the recognition unit 12 (see FIG. 2) is an image capturing device, the recognition unit 12 may acquire the attribute information Ia based on a captured image of the living subject 120. When the recognition unit 12 is a microphone, the recognition unit 12 may acquire the attribute information Ia based on a frequency of voice of the living subject 120.

The state presentation unit 30 (see FIG. 2) may present the state S of the target person 110 based on the attribute information Ia. The state presentation unit 30 may select whether to present or not to present the state S of the target person 110 based on the attribute information Ia. The storage unit 50 may store the attribute information Ia with which the state S may be presented or the attribute information Ia with which the state S is not presented.

The state presentation unit 30 (see FIG. 2) may change a presentation mode of the state S of the target person 110 based on the attribute information Ia. For example, when the state S of the target person 110 is the first state Is-1 (for example, the fatigue state or the like) or the third state Is-3 (for example, the irritated state or the like), and also the living subject 120 is a nurse, the state presentation unit 30 may present the first state Is-1 or the third state Is-3 of the target person 110 to the living subject 120. When the living subject 120 is a nurse, the living subject 120 preferably accurately recognizes the state S of the target person 110. Thus, the state presentation unit 30 may present the first state Is-1 or the third state Is-3 of the target person 110 to the living subject 120.

For example, when the state S of the target person 110 is the first state Is-1 (for example, the fatigue state or the like) or the third state Is-3 (for example, the irritated state or the like), and also the living subject 120 is a visitor, the state presentation unit 30 may discreetly present or may not present the first state Is-1 or the third state Is-3 of the target person 110 to the living subject 120. The visitor in this case may refer to a visitor other than a family member, for example, a visitor such as a friend of the target person 110. A discreet presentation is, for example, a presentation in which the facial expression of the virtual animal displayed on the state presentation unit 30 is set to a blank facial expression or a lackluster facial expression, a presentation without changing the facial expression, or the like. When the living subject 120 is a visitor, since the living subject 120 accurately recognizes the first state Is-1 or the third state Is-3 of the target person 110, the living subject 120 may be shocked. Thus, the state presentation unit 30 may discretely present or may not present the first state Is-1 or the third state Is-3 of the target person 110 to the living subject 120. The state presentation unit 30 may present the first state Is-1 or the third state Is-3 of the target person 110 to a person other than the living subject 120, such as, for example, a nurse.

The determination unit 20 (see FIG. 2) may determine the attribute of the living subject 120 based on the living subject 120 recognized by the recognition unit 12 (see FIG. 2). The information acquisition unit 10 (see FIG. 2) may acquire the attribute information Ia based on the determined attribute. The determination unit 20 may determine whether to present the state S of the target person 110 to the living subject 120 based on the attribute information Ia. When the determination unit 20 determines that the state S is presented, the state presentation unit 30 may present the state S of the target person 110 to the living subject 120.

The determination unit 20 may determine a mode of the presentation of the state S based on the attribute information Ia. The mode of the presentation refers to a presentation of the state S as it is, a discrete presentation of the state S, no presentation of the state S, or the like.

For example, when it is determined that the living subject 120 is a nurse or a family member, the determination unit 20 may determine that the state S is presented as it is. For example, when it is determined that the living subject 120 is a friend or the like of the target person 110, the determination unit 20 may determine whether the state S is discretely presented or is not presented.

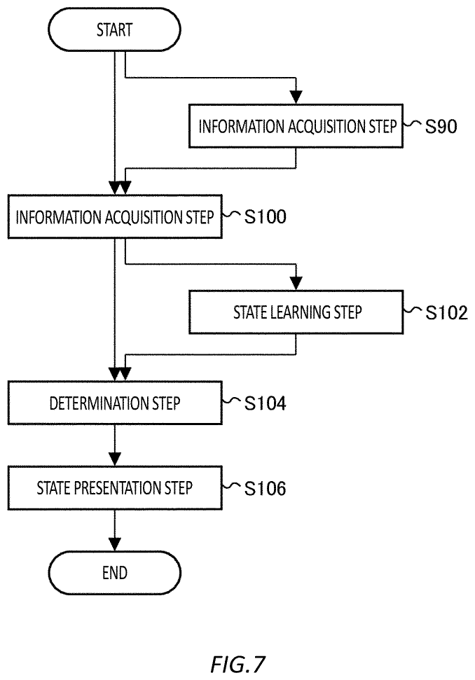

FIG. 7 is a flowchart illustrating an example of an assistance method according to an embodiment of the present invention. The assistance method according to an embodiment of the present invention will be described by using the assistance apparatus 100 illustrated in FIG. 2 as an example. The assistance method includes an information acquisition step S100 and a determination step S104. The assistance method may include an information acquisition step S90, a state learning step S102, and a state presentation step S106.

The information acquisition step S100 is a step for the information acquisition unit 10 to acquire the brain wave information Ib of the target person 110 in a state in which the dialog is difficult in a case where the living subject 120 conducts the engagement En for the target person 110. The determination step S104 is a step for the determination unit 20 to determine the state S of the target person 110 based on the brain wave information Ib acquired in the information acquisition step S100.

The information acquisition step S100 may be a step for the information acquisition unit 10 to further acquire the biological information Ig of the target person 110. The determination step S104 may be a step of determining the state S of the target person 110 based on the brain wave information Ib and the biological information Ig which are acquired in the information acquisition step S100.

The information acquisition step S90 is a step for the information acquisition unit 10 to acquire the brain wave information Ib1 of the target person 110 before the engagement En. The determination step S104 may be a step for the determination unit 20 to generate the state information Is indicating the state S of the target person 110 based on the change from the brain wave information Ib1 before the engagement En to the brain wave information Ib2 after the engagement En and the biological information Ig, and to determine the state S of the target person 110 based on the generated state information Is.

The determination step S104 may be a step for the determination unit 20 to generate the state information Is based on a change from a proportion of the amplitude of the brain wave in a predetermined frequency band to the total amplitude As in the brain wave information Ib1 before the engagement En to a proportion of the amplitude of the brain wave in the frequency band to the total amplitude As in the brain wave information Ib2 after the engagement En and the ratio (LF/HF) of LF to HF in the heartbeat of the target person 110.

The determination step S104 may be a step for the determination unit 20 to generate the state information Is based on the change from the proportion of the amplitude of the brain wave in the predetermined frequency band to the total amplitude As in the brain wave information Ib1 before the engagement En to the proportion of the amplitude of the brain wave in the frequency band to the total amplitude As in the brain wave information Ib2 after the engagement En and a magnitude relationship between the ratio (LF/HF) of LF to HF after the engagement En and a predetermined threshold of the proportion of LF to HF.

The state information Is may include information according to a plurality of states of the target person 110. The determination step S104 may be a step for the determination unit 20 to generate the state information Is according to one state among the plurality of states based on the change from the proportion of the amplitude of the brain wave in the predetermined frequency band to the total amplitude As in the brain wave information Ib1 before the engagement En to the proportion of the amplitude of the brain wave in the frequency band to the total amplitude As in the brain wave information Ib2 after the engagement En and a proportion of the magnitude of the first power spectrum to the magnitude of the second power spectrum.

The state learning step S102 is a step for the state learning unit 40 to generate the engagement inference model 42. The state learning step S102 is a step for the state learning unit 40 to generate, by performing machine learning on the relationship between the brain wave information Ib1 and the engagement En and the state S2, the engagement inference model 42 for making an inference of the engagement En′ for putting the target person 110 into the state S2′ based on the brain wave information Ib1′ and the state S2′.

The determination step S104 may be a step for the determination unit 20 to determine the engagement En for putting the target person 110 into a predetermined state based on the state S of the target person 110 which is inferred in the state learning step S102.

The state presentation step S106 is a step for the state presentation unit 30 to present the state S of the target person 110 which is determined in the determination step S104. The information acquisition step S100 may be a step for the information acquisition unit 10 to further acquire the attribute information Ia indicating the attribute of the living subject 120. The state presentation step S106 may be a step for the state presentation unit 30 to present the state S of the target person 110 based on the attribute information Ia. The state presentation step S106 may be a step for the state presentation unit 30 to change the presentation mode of the state S of the target person 110 based on the attribute information Ia.

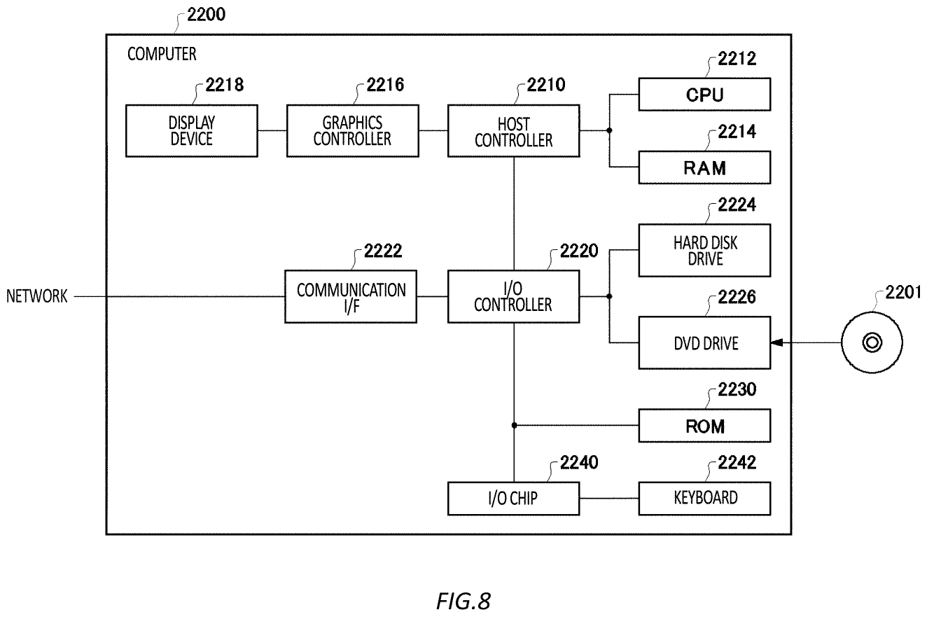

FIG. 8 illustrates an example of a computer 2200 in which the assistance apparatus 100 according to an embodiment of the present invention may be embodied entirely or in part. A program installed in the computer 2200 can cause the computer 2200 to function as an operation associated with the assistance apparatus 100 according to the embodiment of the present invention or as one or more sections of the assistance apparatus 100, or can cause the operation or the one or plurality of sections to be executed, or can cause the computer 2200 to execute each step (see FIG. 7) according to the method of the present invention. The program may be executed by a CPU 2212 in order to cause the computer 2200 to perform particular operations associated with some or all of the blocks in the flowchart (FIG. 7) and the block diagram (FIG. 2) described in the present specification.

The computer 2200 according to an embodiment of the present invention includes a CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input/output unit such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input/output controller 2220. The computer further includes legacy input/output units such as a ROM 2230 and a keyboard 2242. The ROM 2230, the keyboard 2242, and the like are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card.

The ROM 2230 stores a boot program or the like executed by the computer 2200 at the time of activation, or a program depending on the hardware of the computer 2200. The input/output chip 2240 may connect various input/output unit via a parallel port, a serial port, a keyboard port, a mouse port, or the like to the input/output controller 2220.

The program is provided by a computer readable medium such as the DVD-ROM 2201 or

the IC card. The program read from a computer readable medium, installed in the hard disk drive 2224, the RAM 2214, or the ROM 2230 which are also examples of the computer readable medium, and executed by the CPU 2212. The information processing described in these programs is read by the computer 2200 and provides cooperation between the programs and the above-described various types of hardware resources. An apparatus or method may be constituted by realizing the operation or processing of information in accordance with the usage of the computer 2200. For example, when a communication is executed between the computer 2200 and an external device, the CPU 2212 may execute a communication program loaded onto the RAM 2214 to instruct communication processing to the communication interface 2222, based on the processing described in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may execute various types of processing on the data on the RAM 2214. The CPU 2212 may then write back the processed data to the external recording medium.

Various types of information, such as various types of programs, data, tables, and

databases, may be stored in the recording medium to undergo information processing. The CPU 2212 may execute various types of processing on the data read from the RAM 2214, which includes various types of operations, information processing, condition judging, conditional branch, unconditional branch, search or replace of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, read the attribute value of the second attribute stored in the entry, and read a second attribute value to acquire the attribute value of the second attribute associated with the first attribute satisfying the predetermined condition.

The program or software modules described above may be stored in the computer readable media on the computer 2200 or of the computer 2200. A recording medium such as a hard disk or a RAM provided in a server system connected to a dedicated communication network or the Internet can be used as the computer readable media. The program may be provided to the computer 2200 by the recording medium.

While the present invention has been described with the embodiments, the technical scope of the present invention is not limited to the above embodiments. It is apparent to persons skilled in the art that various alterations and improvements can be added to the above-described embodiments. It is also apparent from the description of the claims that embodiments added with such alterations or improvements can be included in the technical scope of the present invention.

The operations, procedures, steps, and stages of each process performed by an apparatus, system, program, and method illustrated in the claims, embodiments, or diagrams can be performed in any order as long as the order is not indicated by “prior to,” “before,” or the like and as long as the outputted from a previous process is not used in a later process. Even if the operation flow is described by using phrases such as “first” or “next” in the scope of the claims, specification, or drawings, it does not necessarily mean that the process must be performed in this order.

### EXPLANATION OF REFERENCES

10: information acquisition unit; 12: recognition unit; 20: determination unit; 30: state presentation unit; 40: state learning unit; 42: engagement inference model; 50: storage unit; 90: control unit; 100: assistance apparatus; 110: target person; 120: living subject; 2200: computer; 2201: DVD-ROM; 2210: host controller; 2212: CPU; 2214: RAM; 2216: graphics controller; 2218: display device; 2220: input/output controller; 2222: communication interface; 2224: hard disk drive; 2226: DVD-ROM drive; 2230: ROM; 2240: input/output chip; and 2242: keyboard.

What is claimed is:

1. An assistance apparatus comprising:
an information acquisition unit which acquires brain wave information of a target person who is in a state in which dialog is difficult in a case where a living subject conducts an engagement for the target person; and
a determination unit which determines a state of the target person based on the brain wave information.

2. The assistance apparatus according to claim 1, wherein
the information acquisition unit further acquires biological information of the target person, and
the determination unit determines the state of the target person based on the brain wave information and the biological information.

3. The assistance apparatus according to claim 2, wherein
the information acquisition unit acquires the brain wave information of the target person before the engagement, and
the determination unit generates state information indicating the state of the target person based on a change from the brain wave information before the engagement to the brain wave information after the engagement and the biological information, and determines the state of the target person based on the state information generated.

4. The assistance apparatus according to claim 3, wherein
the determination unit generates the state information based on a change from a proportion of an amplitude of a brain wave in a predetermined frequency band to a total amplitude in the brain wave information before the engagement to a proportion of the amplitude of the brain wave in the frequency band to the total amplitude in the brain wave information after the engagement and a proportion of a magnitude of a first power spectrum in a heartbeat of the target person to a magnitude of a second power spectrum,
the total amplitude is a sum of amplitudes of an alpha wave, a beta wave, a theta wave, a gamma wave, and a delta wave, and
a frequency band of the second power spectrum is a higher frequency band than a frequency band of the first power spectrum.

5. The assistance apparatus according to claim 4, wherein
the determination unit generates the state information based on the change and a magnitude relationship between the proportion of the magnitude of the first power spectrum to the magnitude of the second power spectrum after the engagement and a predetermined threshold of the proportion of the magnitude of the first power spectrum to the magnitude of the second power spectrum.

6. The assistance apparatus according to claim 4, wherein
the state information includes information according to a plurality of states of the target person, and
the determination unit generates the state information according to one state among the plurality of states based on the change and the proportion of the magnitude of the first power spectrum to the magnitude of the second power spectrum.

7. The assistance apparatus according to claim 6, wherein
the brain wave in the frequency band is at least one of a delta wave, a theta wave, a low alpha wave, or a medium alpha wave.

8. The assistance apparatus according to claim 6, wherein
the brain wave in the frequency band is at least one of a high alpha wave, a low beta wave, a high beta wave, or a gamma wave.

9. The assistance apparatus according to claim 3, further comprising:
a learning unit which generates, by performing machine learning on a relationship between the brain wave information and the engagement and the engagement for putting the state of the target person into a predetermined state, an engagement inference model for making an inference of the engagement for putting the state of the target person into the predetermined state based on the brain wave information and the state of the target person.

10. The assistance apparatus according to claim 1, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

11. The assistance apparatus according to claim 2, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

12. The assistance apparatus according to claim 3, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

13. The assistance apparatus according to claim 4, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

14. The assistance apparatus according to claim 5, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

15. The assistance apparatus according to claim 6, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

16. The assistance apparatus according to claim 7, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

17. The assistance apparatus according to claim 8, further comprising:
a state presentation unit which presents the state of the target person which is determined by the determination unit, wherein
the information acquisition unit further acquires attribute information indicating an attribute of the living subject, and
the state presentation unit presents the state of the target person based on the attribute information.

18. The assistance apparatus according to claim 10, wherein the state presentation unit changes a presentation mode of the state of the target person based on the attribute information.

19. An assistance method comprising:
acquiring, by an information acquisition unit, brain wave information of a target person who is in a state in which dialog is difficult in a case where a living subject conducts an engagement for the target person; and
determining, by a determination unit, a state of the target person based on the brain wave information acquired in the acquiring.

20. A computer readable medium having recorded thereon an assistance program that, when executed by a computer, causes the computer to execute:
acquiring brain wave information of a target person who is in a state in which dialog is difficult in a case where a living subject conducts an engagement for the target person; and
determining a state of the target person based on the brain wave information acquired in the acquiring.
